(19) United States
(12) Patent Application Publication
(10) Pub. No.: US 2025/0160684 A1
(43) Pub. Date: May 22, 2025

(54) REMOTE PATIENT MONITORING

(71) Applicant: Welch Allyn, Inc., Skaneateles Falls, NY (US)

(72) Inventors: Gene J. Wolfe, Pittsford, NY (US); WonKyung McSweeney, Manlius, NY (US); John A. Lane, Venice, FL (US); David E. Quinn, Moravia, NY (US); Tyson B. Whitaker, Arden, NC (US)

(21) Appl. No.: 18/932,277

(22) Filed: Oct. 30, 2024

Related U.S. Application Data
(60) Provisional application No. 63/601,566, filed on Nov. 21, 2023.

(51) Int. Cl. A61B 5/11; A61B 5/00

(52) U.S. Cl. CPC A61B 5/1115; A61B 5/0002; A61B 5/746

(57) ABSTRACT

A system for remotely monitoring a patient. The system performs a base module for remotely monitoring the patient and generates a clinical condition vector using data collected by the base module. The system receives one or more clinical modules from a module store. The one or more clinical modules are received based on the clinical condition vector. The system performs the one or more clinical modules to remotely monitor the patient.

### CROSS REFERENCE TO RELATED APPLICATION

This application claims priority to U.S. Provisional Patent Application No. 63/601,566, filed Nov. 21, 2023, the disclosure of which is hereby incorporated by reference in its entirety.

### BACKGROUND

Presently, there is a shortage of nurses and healthcare professionals. This has led to the development of healthcare delivery models such as virtual nursing and remote patient monitoring. In the case of remote patient monitoring, various monitoring algorithms can be performed to remotely monitor for different types of clinical conditions. However, due to computing capacity and bandwidth constraints, it would be impractical to implement all of the various types of algorithms at the same time when remotely monitoring a patient.

### SUMMARY

In general terms, the present disclosure relates to remote patient monitoring. In one possible configuration, a clinical condition vector is generated from a base module, and one or more clinical modules are received from a module store based on the clinical condition vector for remotely monitoring the patient. Various aspects are described in this disclosure, which include, but are not limited to, the following aspects.

One aspect relates to a system for remotely monitoring a patient, the system comprising: at least one processing device; and at least one computer readable data storage device storing software instructions that, when executed by the at least one processing device, cause the device to: perform a base module to remotely monitor the patient; generate a clinical condition vector using data collected by the base module; receive one or more clinical modules from a module store, the one or more clinical modules received based on the clinical condition vector; and perform the one or more clinical modules to remotely monitor the patient.

Another aspect relates to a method of remotely monitoring a patient, the method comprising: performing a base module to remotely monitor the patient; generating a clinical condition vector using data collected by the base module; receiving one or more clinical modules from a module store, the one or more clinical modules received based on the clinical condition vector; and performing the one or more clinical modules to remotely monitor the patient.

Another aspect relates to a system for providing clinical assessment of a patient, the system comprising: at least one processing device; and at least one computer readable data storage device storing software instructions that, when executed by the at least one processing device, cause the device to: receive a clinical condition vector, the clinical condition vector being a multi-vector score of clinical conditions observed by a base module; match one or more clinical modules to the clinical condition vector; and send the one or more clinical modules to a video analytics system for locally processing data inside a patient environment to remotely monitor the patient.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 illustrates an example of a system for remotely monitoring a patient.

FIG. 2 schematically illustrates an example of a video analytics system that is part of the system of FIG. 1.

FIG. 3 schematically illustrates an example of a module store that is part of the system of FIG. 1.

FIG. 4 schematically illustrates an example of data collected by a base module implemented on the video analytics system of FIG. 2.

FIG. 5 schematically illustrates an example of a method of remotely monitoring the patient that can be performed by the video analytics system of FIG. 2.

FIG. 6 schematically illustrates an example of a method of providing one or more clinical modules that can be performed by the module store of FIG. 3.

FIG. 7 shows an example of a graphical user interface displayed on a device in accordance with an example of the method of FIG. 6.

FIG. 8 schematically illustrates an example of imaging modalities that can be activated and deactivated based on clinical modules on the video analytics system of FIG. 2.

FIG. 9 shows another example of a graphical user interface displayed on the device in accordance with another example of the method of FIG. 6.

FIG. 10 schematically illustrates an example of a method of providing a benchmark comparison of usage of clinical modules stored in the module store of FIG. 3.

FIG. 11 shows another example of a graphical user interface displayed on the device in accordance with an example of the method of FIG. 10.

FIG. 12 schematically illustrates an example of a bed located inside a patient environment shown in FIG. 1.

FIG. 13 schematically illustrates an example of a vital signs monitor located inside the patient environment shown in FIG. 1.

FIG. 14 schematically illustrates an example of an infusion pump located inside the patient environment shown in FIG. 1.

### DETAILED DESCRIPTION

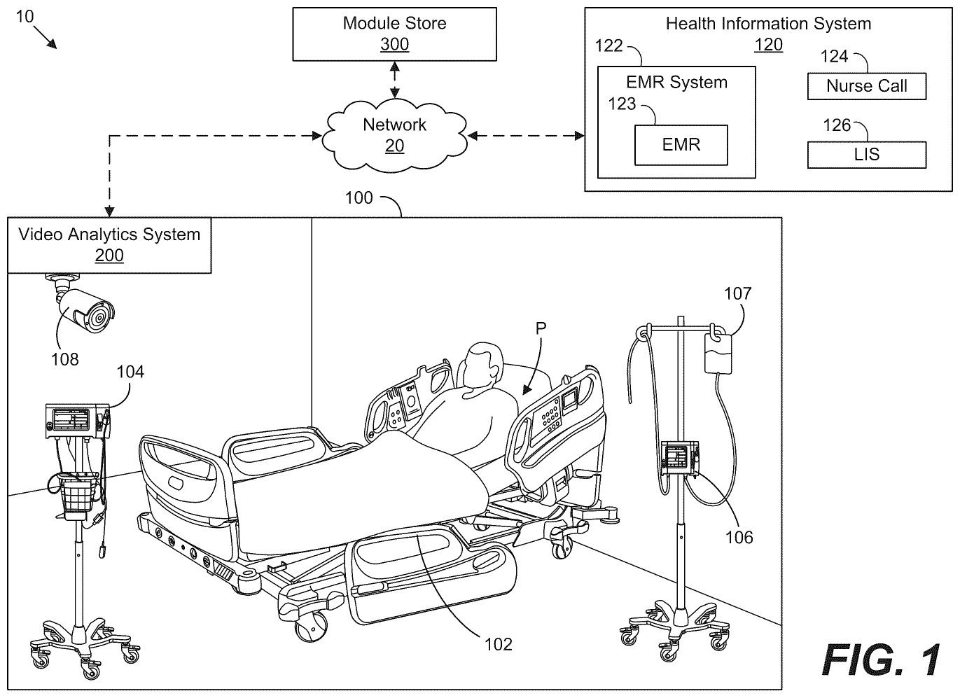

FIG. 1 illustrates an example of a system 10 for remotely monitoring a patient P. The patient P is located inside a patient environment 100. In some examples, the patient environment 100 is a room within a healthcare facility such as a hospital, a nursing home, a long-term care facility, and the like. In other examples, the patient environment 100 is the patient P's home.

The patient environment 100 can include one or more medical devices such as a bed 102, a vital signs monitor 104, and an infusion pump 106. In FIG. 1, the patient P is shown resting on the bed 102. In alternative examples, the patient environment 100 can include additional types of medical devices, or may include fewer medical devices.

The patient environment 100 further includes a data acquisition device 108 that captures image and audio data of the patient environment 100. The data acquisition device 108 is communicatively coupled to a video analytics system 200 such as via a wireless and/or wired connection. The video analytics system 200 continuously performs a base module, while also performing one or more clinical modules for remotely monitoring the patient P. The one or more clinical modules are performed based on a clinical condition vector that is calculated by the base module, and that is updated at predetermined intervals. The term module is used broadly herein to connote a machine-recognizable set of instructions that can be machine-stored, -retrieved, and -executed in a particular situation as disclosed and claimed herein, including but not limited to an algorithm, application, process, procedure, method, subroutine, object, and the like.

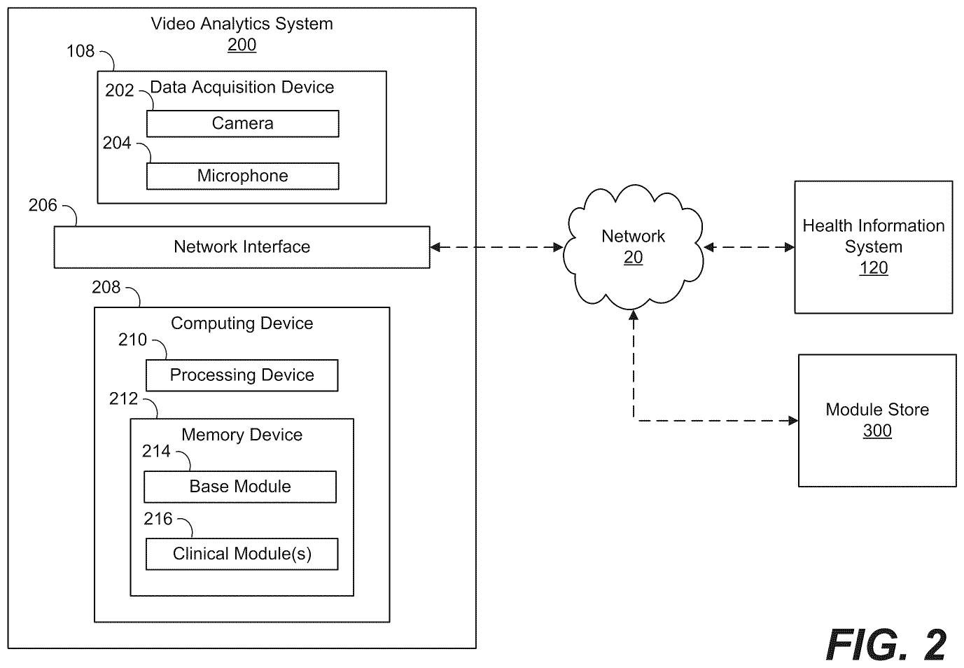

In the example shown in FIG. 1, the video analytics system 200 is illustrated as being located inside the patient environment 100. In this example, the video analytics system 200 processes data locally for remotely monitoring the patient P, while the one or more clinical modules are loaded from a central depository. In some instances, the video analytics system 200 is integrated within one of the devices located inside the patient environment 100. For example, the video analytics system 200 can be integrated within the bed 102, the vital signs monitor 104, the infusion pump 106, or the data acquisition device 108. In alternative examples, the video analytics system 200 can be located outside the patient environment 100 such as in another room or area of the healthcare facility, or outside of the patient P's home. The video analytics system 200 will be described in more detail with reference to FIG. 2.

The system 10 further includes a module store 300 and a health information system (HIS) 120. The video analytics system 200, the module store 300, and the HIS 120 are communicatively coupled via a network 20. In some examples, the video analytics system 200 is also communicatively coupled via the network 20 to one or more devices inside the patient environment 100 such as the bed 102, the vital signs monitor 104, the infusion pump 106, and the data acquisition device 108. The one or more devices inside the patient environment 100 such as the bed 102, the vital signs monitor 104, the infusion pump 106, and/or the data acquisition device 108 can also be communicatively coupled via the network 20 to the HIS 120.

As shown in FIG. 1, the HIS 120 includes an electronic medical record (EMR) system 122 (alternatively termed an electronic health record (EHR)), a nurse call system 124, and a laboratory information system (LIS) 126. The EMR system 122 includes an electronic medical record 123 of the patient P that stores physiological parameter measurements captured by the one or more devices inside the patient environment 100 such as the bed 102, the vital signs monitor 104, the infusion pump 106, and the data acquisition device 108.

The module store 300 stores a plurality of clinical modules that can be performed by the video analytics system 200. The quantity and diversity of the clinical modules can present a significant computing burden on the video analytics system 200 when all clinical modules are performed by the video analytics system 200 simultaneously. Thus, the video analytics system 200 continuously performs the base module to selectively receive one or more clinical modules relevant to the clinical conditions of the patient P which can mitigate computational burden of video processing by the video analytics system 200. Additionally, by selectively receiving clinical modules relevant to the clinical conditions of the patient P, false alarms that result from monitoring for inappropriate clinical states are reduced or eliminated.

The video analytics system 200 continuously performs the base module to generate a clinical condition vector which is determined from the image and audio data captured by the data acquisition device 108. The base module can further generate the clinical condition vector based on data acquired from the HIS 120 including data from one or more of the EMR system 122, the nurse call system 124, and the LIS 126. The base module can further generate the clinical condition vector based on data acquired directly from the bed 102, the vital signs monitor 104, and/or the infusion pump 106. One or more additional clinical modules are acquired by the video analytics system 200 from the module store 300 based on the clinical condition vector. Thereafter, the base module and the one or more additional clinical modules are simultaneously performed by the video analytics system 200 to remotely monitor the patient P.

The video analytics system 200 can activate new clinical modules and deactivate old clinical modules based on changes to the clinical condition vector which is updated by the base module at predetermined intervals. Thus, the base module provides dynamic adaptation of the clinical modules to the current needs of the patient P for virtual nursing support.

Since the data processing is performed locally on the video analytics system 200, which is located inside the patient environment 100, the video analytics system 200 does not need to send large data loads such as videos to the HIS 120 or the module store 300. This can reduce the bandwidth requirements for the network 20, and cause faster communications between the HIS 120, the video analytics system 200, and the module store 300. In some examples, the video analytics system 200 is an edge processing device in the system 10.

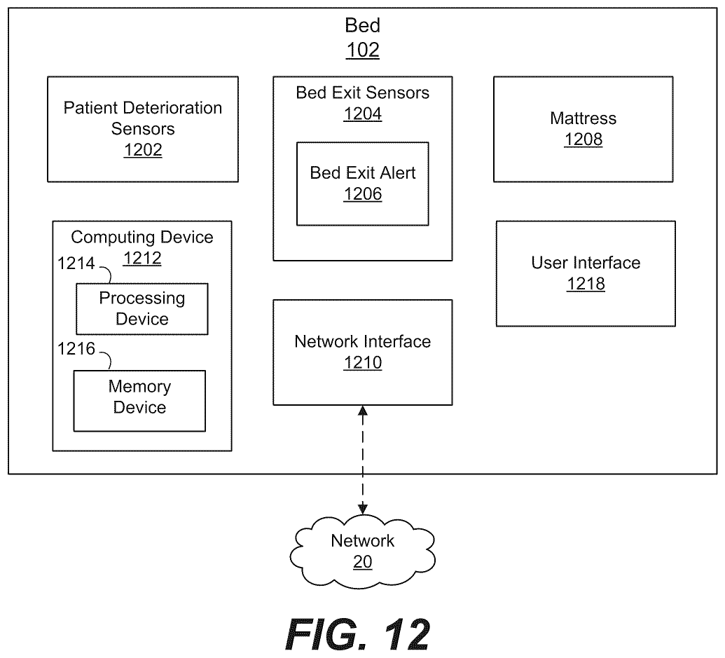

FIG. 12 schematically illustrates an example of the bed 102. Referring now to FIGS. 1 and 12, the bed 102 is a hospital bed that includes patient deterioration sensors 1202 that continuously monitor the patient P's heart rate and respiratory rate in a contact-free manner. The bed 102 includes bed exit sensors 1204 that trigger a bed exit alert 1206, which is configurable to be triggered when the patient P changes position on the bed, moves toward an edge of the bed, or has left bed for fall injury prevention. As further shown in FIG. 12, the bed 102 further includes a mattress 1208 that is controllably inflatable to assist turning the patient P and provide weight-based pressure redistribution for pressure injury prevention.

The bed 102 includes a network interface 1210 that allows the bed 102 to connect to the network 20. The network interface 1210 can include wired interfaces and/or wireless interfaces such as to wirelessly connect to the network 20 through Wi-Fi or other wireless connections, and/or to connect to the network 20 using wired connections such as through an Ethernet or Universal Serial Bus (USB) cable. The bed exit alert 1206 is communicated to the nurse call system 124 via the connection to the network 20, while the patient P's heart rate and respiratory rate are communicated to the video analytics system 200 and/or the EMR system 122 for storage in the EMR 123 of the patient P via the connection to the network 20.

The bed 102 includes a computing device 1212 having a processing device 1214 and a memory device 1216. The bed 102 further includes a user interface 1218 that can include a touchscreen display. The computing device 1212 is communicatively connected to the patient deterioration sensors 1202, the bed exit sensors 1204, the mattress 1208, the network interface 1210, and the user interface 1218. As described further below, in at least some instances, the computing device 1212 controls operation of the patient deterioration sensors 1202, the bed exit sensors 1204, the mattress 1208, the network interface 1210, and the user interface 1218 based on signals received from the video analytics system 200 via the network 20.

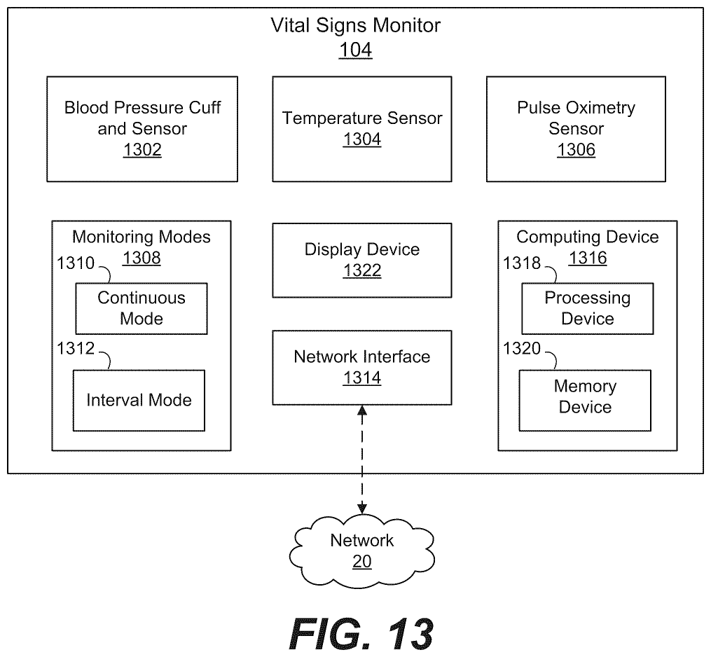

FIG. 13 schematically illustrates an example of the vital signs monitor 104. Referring now to FIGS. 1 and 13, the vital signs monitor 104 includes sensors such as a blood pressure cuff and sensor 1302 for measuring the patient P's blood pressure, a temperature sensor 1304 for measuring the patient P's temperature, and a pulse oximetry sensor 1306 for measuring the patient P's oxygen saturation, pulse, and respiration rate. The vital signs monitor 104 includes monitoring modes 1308 such as a continuous mode 1310 to continuously monitor the vital signs, and an interval mode 1312 to measure the vital signs at predetermined intervals.

The vital signs monitor 104 includes a network interface 1314 that allows the vital signs monitor 104 to connect to the network 20. The network interface 1314 can include wired interfaces and/or wireless interfaces such as to wirelessly connect to the network 20 through Wi-Fi or other wireless connections, and/or to connect to the network 20 using wired connections such as through an Ethernet or Universal Serial Bus (USB) cable. The vital signs measured by the one or more sensors of the vital signs monitor 104 are communicated to the video analytics system 200 and/or the EMR system 122 via the connection to the network 20.

The vital signs monitor 104 includes a computing device 1316 having a processing device 1318 and a memory device 1320. The vital signs monitor 104 further includes a display device 1322 that can include a touchscreen display. The computing device 1316 is communicatively connected to the blood pressure cuff and sensor 1302, the temperature sensor 1304, the pulse oximetry sensor 1306, the network interface 1314, and the display device 1322. As described further below, in at least some instances, the computing device 1316 controls operation of the blood pressure cuff and sensor 1302, the temperature sensor 1304, the pulse oximetry sensor 1306 based on signals received from the video analytics system 200 such as to operate under the continuous mode 1310 or under the interval mode 1312.

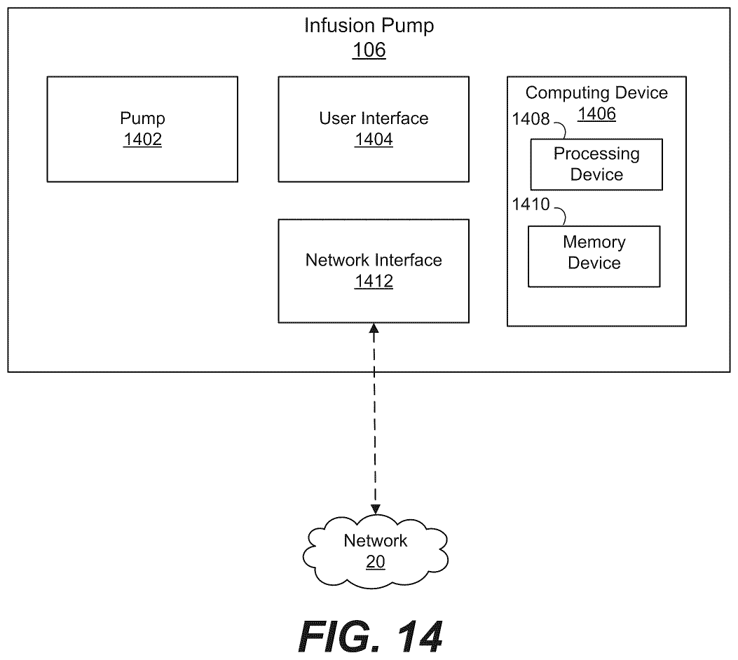

FIG. 14 schematically illustrates an example of the infusion pump 106. Referring now to FIGS. 1 and 14, the infusion pump 106 includes a pump 1402, a user interface 1404, and a computing device 1406 having a processing device 1408 and a memory device 1410. The pump 1402 controls the flow of fluid from a bag 107 (see FIG. 1) for delivery to the patient P. The user interface 1404 can include a touchscreen display. The computing device 1406 is communicatively connected to the pump 1402 and the user interface 1404. As described further below, in at least some instances, the computing device 1406 controls operation of the pump 1402 based on signals received from the video analytics system 200.

The infusion pump 106 includes a network interface 1412 that allows the infusion pump 106 to connect to the network 20. The network interface 1412 can include wired interfaces and/or wireless interfaces such as to wirelessly connect to the network 20 through Wi-Fi or other wireless connections, and/or to connect to the network 20 using wired connections such as through an Ethernet or Universal Serial Bus (USB) cable. A flow rate of liquid pumped by the pump 1402 can be communicated to the video analytics system 200 and/or the health information system 120 via the connection to the network 20, and signals to adjust the flow rate of liquid pumped by the pump 1402 can be received from the video analytics system 200 and/or the health information system 120 via the connection to the network 20.

FIG. 2 schematically illustrates an example of the video analytics system 200. In this illustrative example, the video analytics system 200 includes the data acquisition device 108 (see also FIG. 1) which includes a camera 202 for capturing image data of the patient environment 100, and a microphone 204 for capturing audio data of the patient environment 100.

The video analytics system 200 includes a network interface 206 that allows the video analytics system 200 to connect to the network 20. The network interface 206 can include wired interfaces and wireless interfaces. For example, the network interface 206 can wirelessly connect to the network 20 through cellular network communications, Wi-Fi, or other type of wireless connection. Alternatively, the network interface 206 can connect to the network 20 using wired connections such as through an Ethernet cable, a Universal Serial Bus (USB) cable, or other type of wired connection. As shown in FIG. 2, the connection to the network 20 allows the video analytics system 200 to communicate with the HIS 120 and the module store 300.

The video analytics system 200 includes a computing device 208 having at least one processing device 210 and a memory device 212. The at least one processing device 210 is an example of a processing unit such as a central processing unit (CPU). The at least one processing device 210 can include one or more central processing units (CPUs). In some examples, the at least one processing device 210 includes one or more digital signal processors, field-programmable gate arrays, and/or other types of electronic circuits.

The memory device 212 operates to store data and instructions for execution by the at least one processing device 210. In the example illustrated in FIG. 2, the memory device 212 stores a base module 214 that continuously collects the image and audio data captured by the data acquisition device 108, and additional data acquired from the HIS 120 and/or additional data acquired directly from the bed 102, the vital signs monitor 104, and the infusion pump 106. The base module 214 generates the clinical condition vector at predetermined intervals based on the collected data. Additionally, the memory device 212 temporarily stores one or more clinical modules 216 acquired from the module store 300. The base module 214 and the one or more clinical modules 216 will be described in more detail further below.

The memory device 212 includes computer-readable media, which may include any media that can be accessed by the video analytics system 200. By way of example, computer-readable media include computer readable storage media and computer readable communication media. Computer readable storage media includes volatile and nonvolatile, removable and non-removable media implemented in any device configured to store information such as computer readable instructions, data structures, program modules, or other data. Computer readable storage media can include, but is not limited to, random access memory, read only memory, electrically erasable programmable read only memory, flash memory, and other memory technology, including any medium that can be used to store information that can be accessed by the data acquisition device. The computer readable storage media is non-transitory.

Computer readable communication media embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are within the scope of computer readable media.

The base module 214 continuously collects data relevant to clinical conditions of the patient P. For example, the base module 214 continuously collects the image and audio data captured by the data acquisition device 108 to recognize one or more devices inside the patient environment 100 that are relevant to a clinical condition of the patient P. As an example, when the image data acquired from the data acquisition device 108 shows an insulin pump present inside the patient environment 100, the base module 214 determines that the patient P is diabetic. The image and audio data acquired from the data acquisition device 108 can further show presence of medical specialists or other persons inside the patient environment 100, medical procedures or treatments performed on the patient P inside the patient environment 100, or verbal conversations inside the patient environment 100 such as between the patient P and one or more caregivers to determine the clinical conditions of the patient P.

The base module 214 can also monitor the patient P's ambulation by monitoring the patient P's movements inside the patient environment 100 to determine the clinical conditions of the patient P. The base module 214 can also monitor sounds inside the patient environment 100 such as audible alarms triggered on the bed 102, the vital signs monitor 104, the infusion pump 106, and other devices to determine the clinical conditions of the patient P.

In some instances, data acquired from the HIS 120, the bed 102, the vital signs monitor 104, and the infusion pump 106 supplements the image and audio data acquired from the data acquisition device 108 for determining the clinical conditions of the patient P. For example, the base module 214 receives data such as vital sign measurements, medications, diagnoses, chronic conditions, allergies, and other relevant health information stored in the EMR 123 of the patient P for determining the clinical conditions of the patient P. The base module 214 can also receive data from the nurse call system 124 such as frequency and types of nurse calls for determining the clinical conditions of the patient P. The base module 214 can also receive data from the LIS 126 such as lab test results for determining the clinical conditions of the patient P.

Periodically, such as after a predetermined interval of time elapses, the base module 214 generates the clinical condition vector. As an illustrative example, the clinical condition vector is a multi-vector score of clinical conditions observed from the patient P based on the image and audio data acquired from the data acquisition device 108. The multi-vector score of clinical conditions can also be determined based on data acquired from the HIS 120 (including data acquired from one or more of the EMR system 122, the nurse call system 124, and the LIS 126), and/or data acquired directly from one or more of the bed 102, the vital signs monitor 104, and/or the infusion pump 106 located inside the patient environment 100.

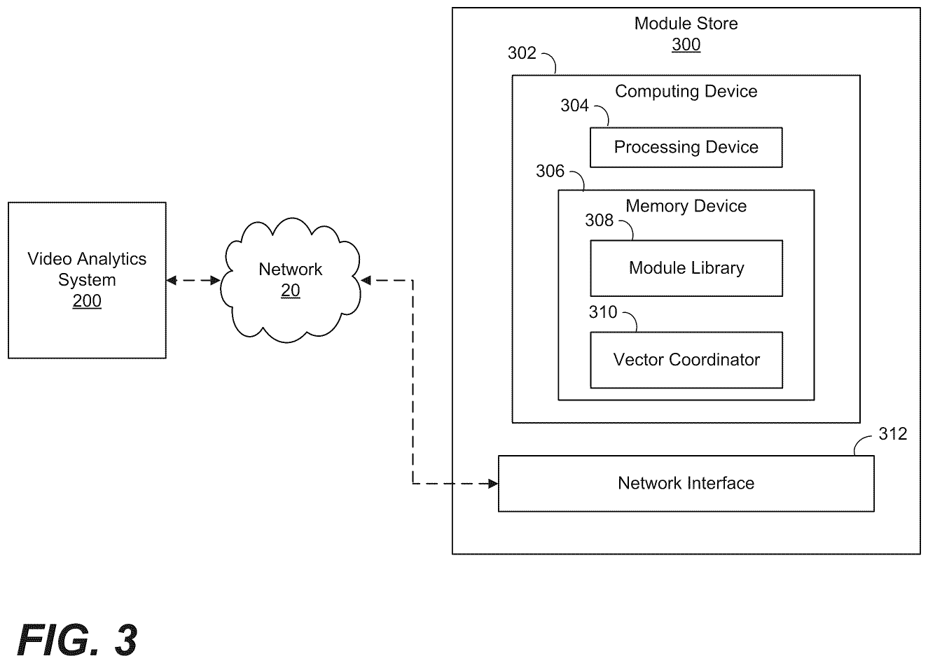

FIG. 3 schematically illustrates an example of the module store 300. As shown in FIG. 3, the module store 300 includes a computing device 302 having at least one processing device 304 and a memory device 306. The computing device 302 can be substantially similar to the computing device 208 of the video analytics system 200, which is described above.

As shown in FIG. 3, the memory device 306 includes a module library 308 that stores a plurality of the clinical modules 216 that can be performed by the video analytics system 200. In some examples, the clinical modules 216 stored in the module library 308 include artificial intelligence and machine learning models. The memory device 306 further includes a vector coordinator 310 that matches one or more of the clinical modules stored in the module library 308 to the clinical condition vector generated by the base module 214.

The module store 300 further includes a network interface 312 that allows the module store 300 to connect to the network 20. The network interface 312 can include wired interfaces and wireless interfaces. For example, the network interface 312 can wirelessly connect to the network 20 through cellular network communications, Wi-Fi, or other type of wireless connection. Alternatively, the network interface 312 can connect to the network 20 using wired connections such as through an Ethernet cable, a Universal Serial Bus (USB) cable, or other type of wired connection. As shown in FIG. 3, the connection to the network 20 allows the module store 300 to communicate with the video analytics system 200.

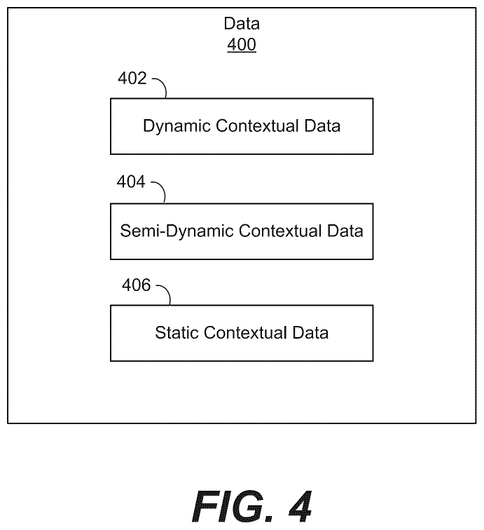

FIG. 4 schematically illustrates an example of data 400 collected by performance of the base module 214 on the video analytics system 200. The base module 214 is continuously performed to monitor clinical conditions of the patient P in the patient environment 100. As shown in FIG. 4, the data 400 includes dynamic contextual data 402, semi-dynamic contextual data 404, and static contextual data 406. The dynamic contextual data 402, semi-dynamic contextual data 404, and static contextual data 406 are used to generate the clinical condition vector, as will be described in more detail further below.

Illustrative examples of the dynamic contextual data 402 can include level of movement by the patient P inside the patient environment 100, location of the patient P inside the patient environment 100, presence of one or more caregivers in the patient environment 100, and other dynamic variables. The dynamic contextual data 402 can be derived from the image and audio data captured inside the patient environment 100 by the data acquisition device 108.

Illustrative examples of the semi-dynamic contextual data 404 can include medications taken by the patient P, Glasgow Coma Scale (GCS) score, pain, agitation, acuity, and other semi-dynamic variables exhibited by the patient P. The semi-dynamic contextual data 404 can be derived from the image and audio data captured by the data acquisition device 108, and/or can be derived from the data acquired over the network 20 from the HIS 120.

Illustrative examples of the static contextual data 406 can include comorbidities, diagnoses, and other chronic conditions of the patient P. The static contextual data 406 can be derived from the data acquired over the network 20 from the HIS 120. In particular, the static contextual data 406 can be derived from the data stored in the EMR 123 of the patient P.

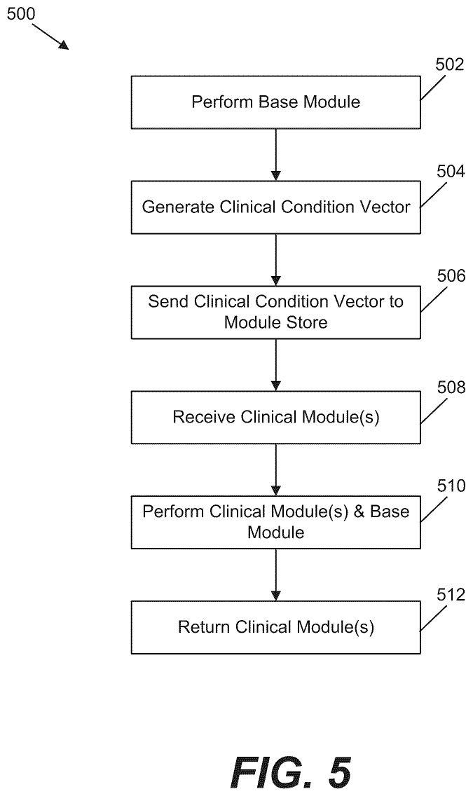

FIG. 5 schematically illustrates an example of a method 500 of remotely monitoring the patient P. In some examples, the method 500 is performed by the video analytics system 200.

The method 500 includes an operation 502 of performing the base module 214. The base module 214 can include collecting the dynamic contextual data 402, the semi-dynamic contextual data 404, and the static contextual data 406 over a predetermined period of time. For example, the base module 214 can include observing and aggregating data relevant to clinical conditions of the patient P such as image data collected by the camera 202 of the data acquisition device 108 that identifies equipment in operation around the patient P and/or medical activities performed around the patient P. For example, the image data collected by the camera 202 can identify medical equipment in operation inside the patient environment 100 and/or medical activities performed inside the patient environment 100 such as a presence of medical personnel, medical procedures and treatments performed on the patient P, and the like.

The base module 214 also receives a data feed from the HIS 120. For example, the data feed from the HIS 120 can include vital sign measurements, medications, diagnoses, chronic conditions, allergies, and other relevant health information of the patient P.

After a predetermined interval of time, the method 500 includes an operation 504 of generating a clinical condition vector based on the data observed and aggregated by performance of the base module 214 in operation 502. In some instances, the clinical condition vector is a multi-vector score of clinical conditions exhibited by the patient P. For example, the patient P may simultaneously experience multiple clinical conditions at the same time.

The method 500 includes an operation 506 of sending the clinical condition vector to the module store 300. As described above, the clinical condition vector can be sent by the video analytics system 200 to the module store 300 over the network 20. When the module store 300 receives the clinical condition vector, the module store 300 searches through the module library 308 to match one or more clinical modules to the clinical condition vector.

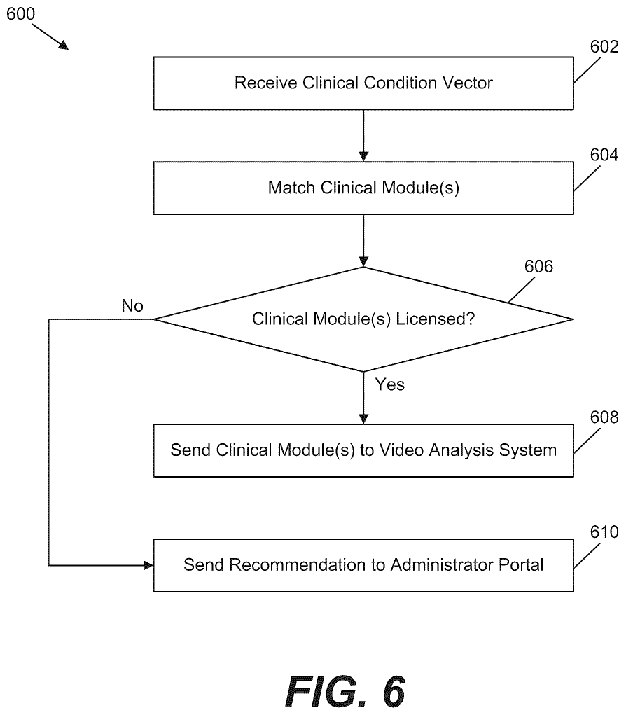

The module store 300 identifies one or more clinical modules stored in the module library 308 that have characteristics matching the clinical condition vector, and that are available for use by the video analytics system 200. For example, the module store 300 can match the clinical condition vector to one or more clinical modules licensed for use by the video analytics system 200. Additional details of the operations performed by the module store 300 are described with reference to the method 600 illustrated in FIG. 6.

Still referring to FIG. 5, the method 500 includes an operation 508 of receiving the one or more clinical modules 216 from the module store 300. As described above, the one or more clinical modules can be received from the module store 300 over the network 20.

The method 500 includes an operation 510 of performing the one or more clinical modules 216 received from the module store 300 in operation 508, while continuing to perform the base module 214. The method 500 can include repeating the operations 504-510 such that the base module 214 is continuously performed to update the clinical condition vector at predetermined intervals such that new and/or different clinical modules can be performed by the video analytics system 200 as the clinical conditions of the patient P change. Thus, the method 500 provides dynamic adaptation of the clinical modules performed by the video analytics system 200 based on current needs for virtual nursing support. By using the base module 214 that can process and interpret the video and/or audio data captured by the data acquisition device 108 in real-time, a variety of different clinical workflows can be performed on the video analytics system 200, where each clinical workflow is powered independently by a clinical module 216 pulled from the module store 300 over the network 20.

The method 500 can further include an operation 512 of returning the one or more clinical modules 216 to the module store 300. Operation 512 can occur when the clinical condition vector calculated by the base module 214 changes due to changed clinical conditions of the patient P detected from the data captured by the data acquisition device 108 and/or the data acquired from the HIS 120. In such instances, the one or more clinical modules 216 are no longer needed for remotely monitoring the patient P. In further examples, operation 512 can include returning the one or more clinical modules 216 to the module store 300 when the patient P leaves the patient environment 100 such as when the patient P is discharged from the healthcare facility. In some examples, operation 512 includes deleting the one or more clinical modules 216 from the memory of the video analytics system 200.

The method 500 has several benefits. First, the method 500 allows the video analytics system 200 to be tailored to specific clinical needs and patient populations by selecting and pulling the clinical modules 216 that are most relevant to a given patient situation. For example, by performing the method 500, a first type of clinical module 216 can be pulled from the module store 300 over the network 20 for monitoring vital signs in a post-surgical patient environment, whereas a second type of clinical module 216 can be pulled from the module store 300 over the network 20 for monitoring a patient with a chronic medical condition.

Additionally, the method 500 allows for use of clinical modules 216 that are independent of one another which can help to improve the accuracy of the system 10 overall. For example, the clinical modules 216 can provide multiple perspectives and diagnostic approaches, which can reduce a risk of false positives or false negatives, and increase a likelihood that the system 10 will accurately identify changes in the patient P's clinical state.

FIG. 6 schematically illustrates an example of a method 600 of providing the one or more clinical modules 216 to the video analytics system 200. In some examples, the method 600 is performed by the module store 300. In particular, the method 600 can be performed by the vector coordinator 310 when installed on the module store 300.

The method 600 includes an operation 602 of receiving the clinical condition vector from the video analytics system 200. As described above, the clinical condition vector can be received from the video analytics system 200 over the network 20.

The method 600 includes an operation 604 of matching one or more clinical modules 216 to the clinical condition vector. As described above, the clinical condition vector can be a multi-vector score of clinical conditions calculated by the base module 214 using the dynamic contextual data 402, the semi-dynamic contextual data 404, and the static contextual data 406. In some examples, operation 604 can include matching metadata associated with the clinical modules 216 to the multi-vector scores included in the clinical condition vector.

The module store 300 is a rule-based system that can include trigger rules based on the clinical condition vectors generated by the base module 214. The vector coordinator 310 can include one or more machine learning models that match the clinical condition vectors to the trigger rules. In some examples, the one or more machine leaning models include decision tree learning such as a classification or regression decision tree that is used as a predictive model to draw conclusions about a set of observations (e.g., the clinical condition vector) acquired from the base module 214. The clinical condition vector can include a multidimensional array having one or more types of binary, continuous, or conditional data types. In some examples, the vector coordinator 310 produces a binary decision such as match or no-match. Alternatively, the vector coordinator 310 can calculate a continuous value that represents a matching strength between the clinical condition vector and one or more clinical modules 216 when appropriate.

The method 600 can include an operation 606 of determining whether the one or more clinical modules 216 matched in operation 604 are licensed for use on the video analytics system 200. As an illustrative example, an administrator of the video analytics system 200 such as a healthcare facility (e.g., a hospital, nursing home, long-term care facility, and the like) can purchase licenses for each clinical module 216 stored in the module library 308 of the memory device 306. The licenses can be purchased based on a quantity (e.g., 500 licenses), a time duration (e.g., 1,000 hours), or some other type of metric. Additionally, the administrator of the video analytics system 200 may purchase different quantities or durations of licenses for the clinical modules 216 (e.g., the administrator of the video analytics system 200 can purchase a quantity of 500 licenses for a first clinical module, a quantity of 1,000 licenses for a second clinical module, and a quantity of 750 licenses for a third clinical module).

When the one or more clinical modules 216 matched in operation 604 are licensed for use on the video analytics system 200 (i.e., “Yes” in operation 606), the method 600 proceeds to an operation 608 of sending the one or more clinical modules 216 to the video analytics system 200. In some examples, the module store 300 can request confirmation from a user such as a caregiver on whether to implement the one or more clinical modules 216.

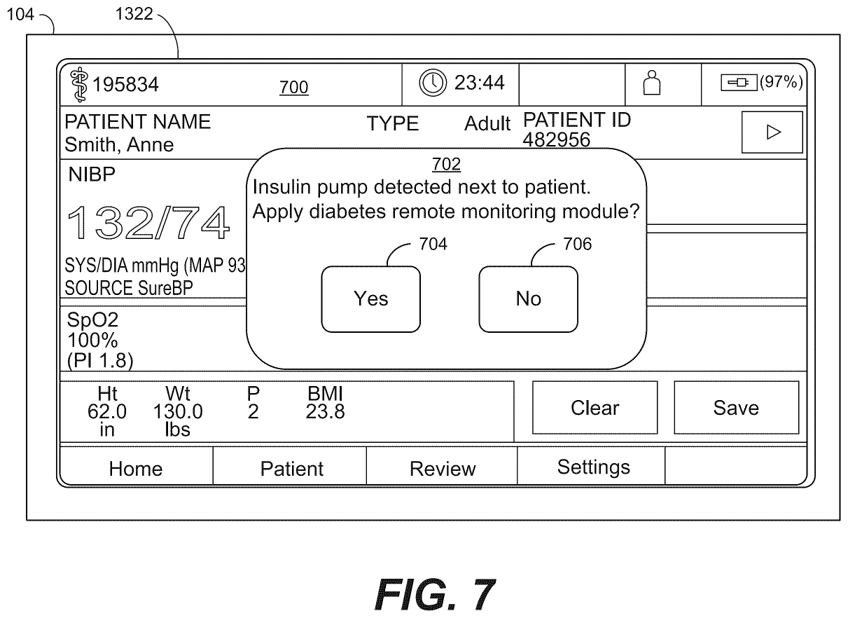

FIG. 7 shows an example of a graphical user interface 700 displayed in accordance with an example of the method 600. In FIG. 7, the graphical user interface 700 is shown displayed on the display device 1322 of the vital signs monitor 104. In other examples, the graphical user interface 700 can be displayed on the user interface 1218 of the bed 102 (see FIG. 12) and/or on the user interface 1404 of the infusion pump 106 (see FIG. 14). In further examples, the graphical user interface 700 can be displayed on a display device outside of the patient environment 100 such as on a display device located in a nurses' station or on an administrator portal that is operated by an administrator of the healthcare facility.

The graphical user interface 700 includes a prompt 702 that includes a message “Insulin pump detected next to patient. Apply diabetes remote monitoring application?”, a first option 704 to accept implantation of the diabetes remote monitoring application by the video analytics system 200, and a second option 706 to decline implantation of the diabetes remote monitoring application by the video analytics system 200. When the user selects the first option 704, the video analytics system 200 can begin to remotely monitor the patient P using the diabetes remote monitoring application. Further, the module store 300 can register use of a license of the diabetes remote monitoring application by the video analytics system 200. As an example, the license is used per patient (i.e., 500 licenses for use on 500 patients). Alternatively, the license can be used per time metric (i.e., 1,000 hours of total usage).

A clinical module 216 when implemented on the video analytics system 200 can affect operations of the video analytics system 200. For example, the base module 214 is a computationally light module that runs in the background to keep track of the one or more clinical conditions of the patient P such as by continuously updating the clinical condition vector based on the dynamic contextual data 402, the semi-dynamic contextual data 404, and the static contextual data 406. In contrast, the clinical module 216 can cause activation or deactivation of one or more features on the video analytics system 200, alter a trigger frequency of the one or more features on the video analytics system 200 (e.g., continuously performed, or performed every 10 minutes, every 60 minutes, and the like), alter a duration of one or more settings on the on the video analytics system 200 (e.g., until a next triggering event, a set amount of time, and the like), alter a level or severity of alerts generated by the on the video analytics system 200 (e.g., record event, issue warning to patient P, send alert to nurse call system 124, issue code blue alarm), or alter a trigger of audio cues on the video analytics system 200.

In some examples, the base module 214 and/or the clinical module 216 can provide one or more types of operational analytics such as tracking equipment use, time spent by caregivers and other staff members inside the patient environment 100, usage of materials and other consumables, and the like. The base module 214 and/or the clinical module 216 can provide metrics that can be used to benchmark the operation of the patient environment 100 with other patient environments within the same healthcare facility or different healthcare facilities.

The video analytics system 200 activates detection features for one or more clinical modules 216 that require heavier computing and energy loads than the detection features of the base module 214 only when the detection features of the one or more clinical modules 216 are relevant for remotely monitoring the clinical conditions of the patient P. This can reduce the computing loads and energy consumption required to operate the video analytics system 200, thereby reducing a computational burden of video processing inside the patient environment 100.

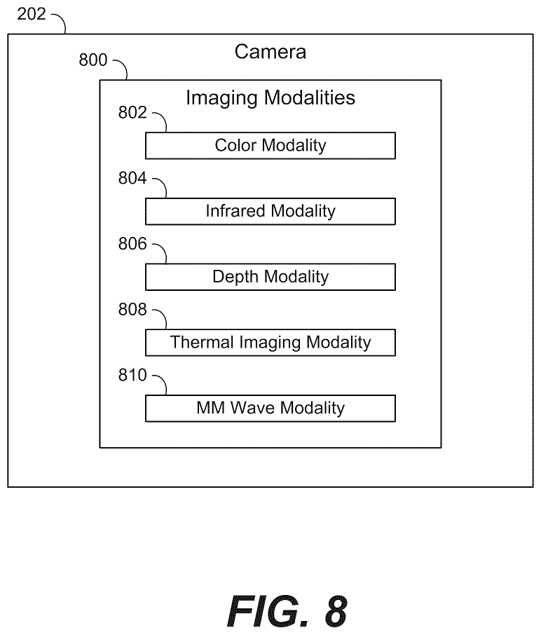

FIG. 8 schematically illustrates an example of imaging modalities 800 of the camera 202 that can be activated and deactivated based on a clinical module 216 implemented on the video analytics system 200. In the example shown in FIG. 8, the imaging modalities 800 include a color imaging modality 802 to capture a color video of the patient environment 100, an infrared imaging modality 804 to capture an infrared video of the patient environment 100, a depth imaging modality 806 to capture a depth video of the patient environment 100, and a thermal imaging modality 808 to capture a thermal image video of the patient environment 100. The imaging modalities 800 can further includes a millimeter wave modality 810 to capture millimeter wave data of the patient P that can be used to measure one or more vital signs of the patient P such as respiration rate and/or heart rate based on motions of the patient P's chest. The imaging modalities 800 are provided by way of illustrative example such that the data acquisition device 108 can include additional types of modalities, or can include fewer types of modalities.

A clinical module 216 when implemented on the video analytics system 200 causes one or more of the imaging modalities 800 to be activated and/or one or more of the imaging modalities 800 to be deactivated based on the clinical module 216. As an illustrative example, a clinical module 216 can require thermal imaging such that the thermal imaging modality 808 is activated, while the other modalities are deactivated. When a different type of clinical module is implemented on the video analytics system 200 based on changes to the clinical condition vector calculated by the base module 214, the thermal imaging modality 808 can be deactivated, and another type of imaging modality can be activated on the camera 202.

A clinical module 216 when implemented on the video analytics system 200 can also affect operations of other devices located inside the patient environment 100. For example, a clinical module 216 when implemented on the video analytics system 200 can cause adjustment of the bed exit alert 1206 on the bed 102 such that the bed exit alert 1206 is triggered based on a different type of event for fall injury prevention (e.g., when the patient P changes position on the bed, moves toward an edge of the bed, or has left bed).

As another example, a clinical module 216 when implemented on the video analytics system 200 causes adjustment of the monitoring modes 1308 on the vital signs monitor 104 such as switching from the interval mode 1312 to the continuous mode 1310, or vice-versa.

As another example, a clinical module 216 when implemented on the video analytics system 200 causes adjustment of a flow rate of fluids, medications, and/or nutrients infused into the patient P's circulatory system by the pump 1402 of the infusion pump 106.

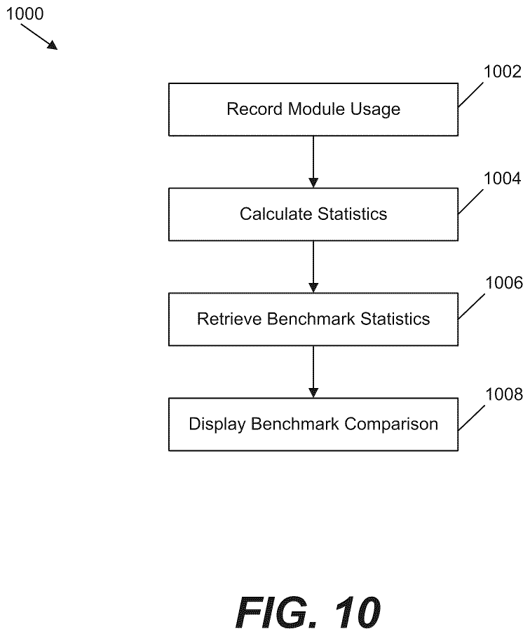

Referring back to FIG. 6, when the one or more clinical modules 216 matched in operation 604 are not licensed for use on the video analytics system 200 (i.e., “No” in operation 606), the method 600 proceeds to an operation 610 of sending a recommendation to purchase a license for the one or more clinical modules 216 matched in operation 604. Operation 610 can include sending the recommendation to an administrator portal where the recommendation can be aggregated and directed to an end point user such as an administrator or clinician who possesses appropriate decision making authority. The recommendation can be aggregated with additional information such as benchmark statistics (see FIG. 10) to facilitate the administrator to decide on whether to purchase a license for the one or more clinical modules. Such additional information can include display of a comparison of a quantity of implementations for each of the clinical modules, a total time duration of usage for each of the clinical modules, and/or an average per patient admission that each of the clinical modules is used in a benchmark healthcare facility such as a healthcare facility having a similar size, medical specialty, or geography of the healthcare facility where the video analytics system 200 is being used.

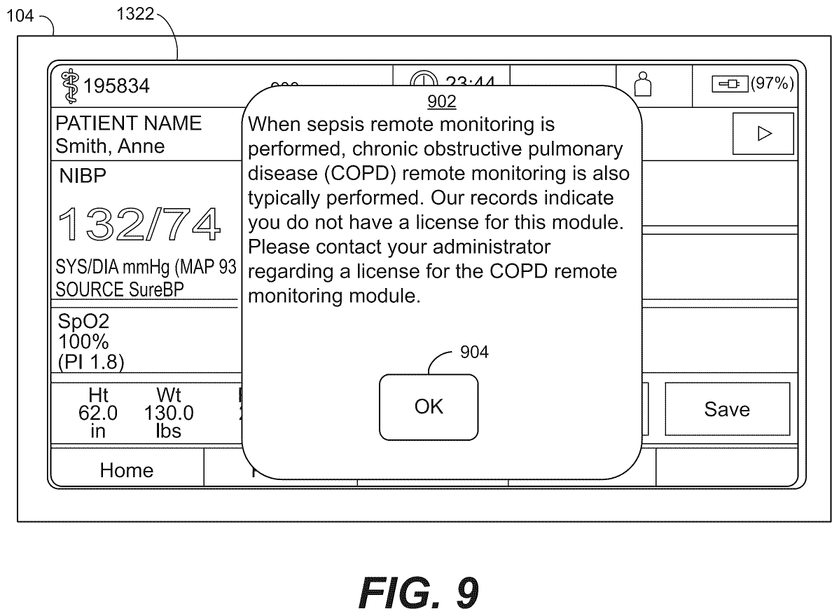

FIG. 9 shows another example of a graphical user interface 900 displayed in accordance with another example of the method 600. In FIG. 9, the graphical user interface 900 is shown displayed on the display device 1322 of the vital signs monitor 104. In other examples, the graphical user interface 900 can be displayed on the user interface 1218 of the bed 102, on the user interface 1404 of the infusion pump 106, or on another display device.

The graphical user interface 900 includes a prompt 902 that includes a recommendation “When sepsis remote monitoring is performed, chronic obstructive pulmonary disease (COPD) remote monitoring is also typically performed. Our records indicate you do not have a license for this module. Please contact your administrator regarding a license for the COPD remote monitoring module.”, and an “OK” icon 904 to close the prompt 902.

FIG. 10 schematically illustrates an example of a method 1000 of providing a benchmark comparison of usage of the clinical modules 216 stored in the module store 300. In some examples, the method 1000 can be performed by the module store 300.

The method 1000 includes an operation 1002 of recording module usage across the healthcare facility where the patient environment 100 is located. For example, operation 1002 can include recording each time a clinical module 216 is implemented on a video analytics system 200 across multiple patient environments in the healthcare facility.

The method 1000 includes an operation 1004 of calculating one or more statistics based on the usage of each of the clinical modules 216 in the healthcare facility. For example, operation 1004 can include calculating a quantity of implementations for each of the clinical modules 216, a total time duration of usage for each of the clinical modules 216, and/or an average per patient admission that each of the clinical modules 216 has been implemented.

The method 1000 includes an operation 1006 of retrieving benchmark statistics from other healthcare facilities that share one or more characteristics with the healthcare facility where the patient environment 100 is located. For example, operation 1006 can include retrieving benchmark statistics from other healthcare facilities that have the same size or capacity of the healthcare facility where the patient environment 100 is located, that have a similar geographic area or region of the healthcare facility where the patient environment 100 is located (e.g., Northeastern United States), and/or that have a similar specialty as the healthcare facility where the patient environment 100 is located (e.g., pediatric healthcare).

The method 1000 includes an operation 1008 of displaying a comparison of the benchmark statistics retrieved in operation 1006 to the statistics calculated in operation 1004. For example, operation 1008 can include displaying a comparison of a quantity of implementations for each of the clinical modules 216, a total time duration of usage for each of the clinical modules 216, and/or an average per patient admission that each of the clinical modules 216.

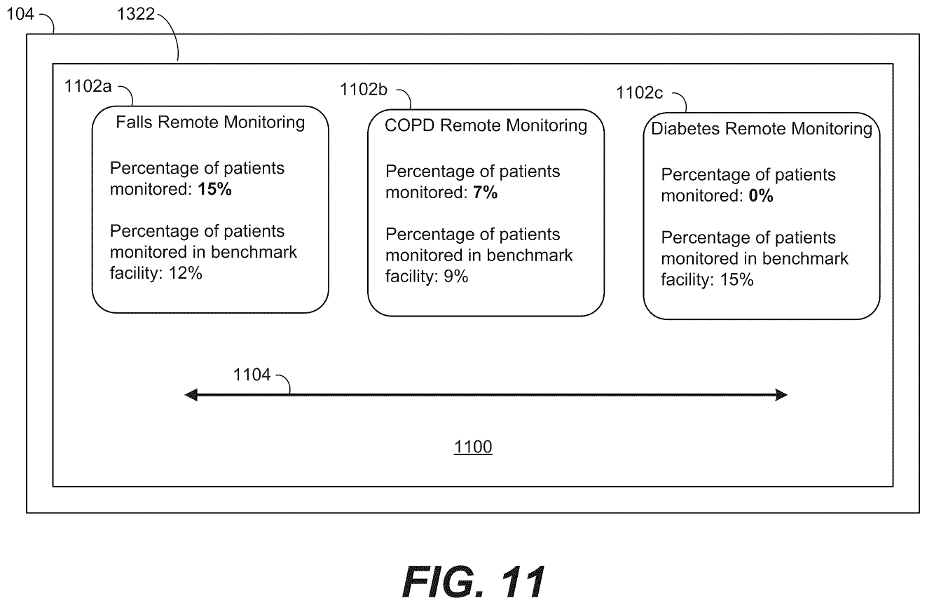

FIG. 11 shows an example of a graphical user interface 1100 displayed in accordance with an example of the method 1000. In FIG. 11, the graphical user interface 1100 is shown displayed on the display device 1322 of the vital signs monitor 104. In other examples, the graphical user interface 1100 can be displayed on the user interface 1218 of the bed 102, on the user interface 1404 of the infusion pump 106, or on another display device.

The graphical user interface 1100 includes one or more statistical comparisons 1102 of usage of the clinical modules 216 between the healthcare facility where the patient environment 100 is located and a benchmark healthcare facility such as a healthcare facility having similar size, location, and/or specialty. The graphical user interface 1100 can further include a scroll bar 1104 allowing the user to scroll through additional statistical comparisons.

In the illustrative example shown in FIG. 11, a first statistical comparison 1102a shows that a falls remote monitoring module was implemented for 15% of the patients admitted to the healthcare facility where the patient environment 100 is located, while the falls remote monitoring module was implemented for 12% of the patients admitted to the benchmark healthcare facility. The percentage for the falls remote monitoring module is higher for the healthcare facility where the patient environment 100 is located such that it is highlighted in green to show that it is being used more frequently than in the benchmark healthcare facility.

In FIG. 11, a second statistical comparison 1102b shows that a COPD remote monitoring module was implemented for 7% of the patients admitted to the healthcare facility where the patient environment 100 is located, while the COPD remote monitoring module was implemented for 9% of the patients admitted to the benchmark healthcare facility. Since the percentage for the COPD remote monitoring module is lower for the healthcare facility where the patient environment 100 is located, it is highlighted in red to indicate that additional licenses for this module should be purchased to reach the usage of the benchmark facility.

In FIG. 11, a third statistical comparison 1102c shows that a diabetes remote monitoring module was implemented for 0% of the patients admitted to the healthcare facility where the patient environment 100 is located (perhaps due to the healthcare facility not purchasing licenses for this module), while the diabetes remote monitoring module was implemented for 15% of the patients admitted to the benchmark healthcare facility. Since, the percentage for the diabetes remote monitoring module is zero for the healthcare facility where the patient environment 100 is located, it is highlighted in red to indicate that licenses for this module should be purchased to reach the usage of the benchmark facility.

The various embodiments described above are provided by way of illustration only and should not be construed to be limiting in any way. Various modifications can be made to the embodiments described above without departing from the true spirit and scope of the disclosure.

What is claimed is:

1. A system for remotely monitoring a patient, the system comprising:
at least one processing device; and
at least one computer readable data storage device storing software instructions that, when executed by the at least one processing device, cause the device to:
perform a base module to remotely monitor the patient;
generate a clinical condition vector using data collected by the base module;
receive one or more clinical modules from a module store, the one or more clinical modules received based on the clinical condition vector; and
perform the one or more clinical modules to remotely monitor the patient.

2. The system of claim 1, wherein the instructions, when executed by the at least one processing device, further cause the system to:
activate one or imaging modalities based on the one or more clinical modules.

3. The system of claim 1, wherein the instructions, when executed by the at least one processing device, further cause the system to:
simultaneously perform the one or more clinical modules while continuously performing the base module for remotely monitoring the patient.

4. The system of claim 1, wherein the instructions, when executed by the at least one processing device, further cause the system to:
return the one or more clinical modules to the module store due to changes in the clinical condition vector calculated by the base module.

5. The system of claim 1, wherein the data collected by the base module includes image data identifying equipment in operation around the patient, presence of medical personnel around the patient, and medical activities performed on the patient.

6. The system of claim 1, wherein the data collected by the base module includes vital sign measurements, medications, diagnoses, and chronic conditions of the patient.

7. The system of claim 1, wherein the clinical condition vector is a multi-vector score of clinical conditions observed by the base module.

8. A method of remotely monitoring a patient, the method comprising:
performing a base module to remotely monitor the patient;
generating a clinical condition vector using data collected by the base module;
receiving one or more clinical modules from a module store, the one or more clinical modules received based on the clinical condition vector; and
performing the one or more clinical modules to remotely monitor the patient.

9. The method of claim 8, further comprising:
activating one or imaging modalities based on the one or more clinical modules.

10. The method of claim 8, further comprising:
simultaneously performing the one or more clinical modules while continuously performing the base module for remotely monitoring the patient.

11. The method of claim 8, further comprising:
returning the one or more clinical modules to the module store due to changes in the clinical condition vector calculated by the base module.

12. The method of claim 8, wherein the data collected by the base module includes image data identifying equipment in operation around the patient, presence of medical personnel around the patient, and medical activities performed on the patient.

13. The method of claim 8, wherein the data collected by the base module includes vital sign measurements, medications, diagnoses, and chronic conditions of the patient.

14. The method of claim 8, wherein the clinical condition vector is a multi-vector score of clinical conditions observed by the base module.

15. A system for providing clinical assessment of a patient, the system comprising:
at least one processing device; and
at least one computer readable data storage device storing software instructions that, when executed by the at least one processing device, cause the device to:
receive a clinical condition vector, the clinical condition vector being a multi-vector score of clinical conditions observed by a base module;
match one or more clinical modules to the clinical condition vector; and
send the one or more clinical modules to a video analytics system for locally processing data inside a patient environment to remotely monitor the patient.

16. The system of claim 15, wherein the instructions, when executed by the at least one processing device, further cause the system to:
display a prompt on a graphical user interface, the prompt including a first option to accept implantation of the one or more clinical modules, and a second option to decline implantation of the one or more clinical modules.

17. The system of claim 15, wherein the instructions, when executed by the at least one processing device, further cause the system to:
display usage comparisons of the one or more clinical modules between a healthcare facility where the patient is admitted and a benchmark healthcare facility.

18. The system of claim 15, wherein the one or more clinical modules activate one or imaging modalities on the video analytics system.

19. The system of claim 15, wherein the one or more clinical modules cause selection of a triggering event for a bed exit alert on a bed where the patient is resting.

20. The system of claim 15, wherein the one or more clinical modules cause selection of a continuous mode or an interval mode for one or more sensors on a vital signs monitor capturing vital sign measurements of the patient.
